SEQUENCE LISTING (6 sequences)

SEQ ID NO: 1 - DNA, 20 bp, Artificial Sequence
  synthetic primer
gtgttgttcccactaataat

SEQ ID NO: 2 - DNA, 19 bp, Artificial Sequence
  synthetic primer
atgcatgaaggggctggac

SEQ ID NO: 3 - DNA, 20 bp, Artificial Sequence
  synthetic primer
tcaacccataataagagatc

SEQ ID NO: 4 - DNA, 19 bp, Artificial Sequence
  synthetic primer
catcctattggatgctcgc

SEQ ID NO: 5 - DNA, 20 bp, Artificial Sequence
  synthetic primer
atccgacttttataaggacg

SEQ ID NO: 6 - DNA, 20 bp, Artificial Sequence
  synthetic primer
tgatgttaccaacgaagatg

(19) United States
(12) Patent Application Publication
(10) Pub. No.: US 2012/0214867 A1
(43) Pub. Date: Aug. 23, 2012

(54) Bacteriophage T4 Nanoparticles for Eukaryotic Delivery

(75) Inventors: Jinny Lin Liu, Ellicott, MD (US); Kelly L. Robertson, Arlington, VA (US); Carissa M. Soto, Alexandria, VA (US)

(73) Assignee: The Government of the United States of America, as represented by the Secretary of the Navy, Washington, DC (US)

(21) Appl. No.: 13/396,110

(22) Filed: Feb. 14, 2012

Related U.S. Application Data
(60) Provisional application No. 61/444,416, filed on Feb. 18, 2011.

(51) Int. Cl. A61K 48/00; C12N 5/071; G01N 21/64; B82Y 5/00

(52) U.S. Cl. 514/44 R; 435/6.1; 435/375; 977/773; 977/916

(57) ABSTRACT

A T4 nanoparticle is a non-infectious, tail-less variant of a T4 bacteriophage. In one embodiment, eukaryotic cells are labeled with dyed T4 nanoparticles, wherein each dyed T4 nanoparticle comprises at least 350 dye molecules covalently bound thereto. In another embodiment, T4 nanoparticles are used to deliver exogenous DNA to eukaryotic cells for protein expression therein. It is contemplated that T4 nanoparticles may be used to deliver other exogenous material to eukaryotic cells.

### CROSS-REFERENCE TO RELATED APPLICATIONS

This Application claims the benefit of U.S. Provisional Application No. 61/444,416 filed on Feb. 18, 2011, incorporated herein by reference.

### BACKGROUND

Viral scaffolds can be tuned as desired through chemical surface modulation or genetic modification, for example for display on the scaffold surface. Among these, T4 nanoparticles (NPs) have attracted attention due to their accessibility and properties.

As described by Archer and Liu, Sensors 9 (2009), 6298-6311, interest in the use of phages and particularly bacteriophage T4 as a nano-material has recently increased, due to its flexible display system. For material and sensor applications, non-infectious T4 nanoparticles, consisting only of the capsid, or the capsid and the whiskers, can be synthesized by deletion of the tail through genetic engineering. This deletion can be accompanied with surface engineering to express capture moieties specific for a particular target leading to functional T4 nanoparticles for use as biorecognition elements in sensor devices. See also US Patent Application Publication No. 2002/0025515.

### BRIEF SUMMARY

In one embodiment, a method of labeling eukaryotic cells includes providing dyed T4 nanoparticles, wherein each dyed T4 nanoparticle comprises at least 350 dye molecules covalently bound thereto, and contacting eukaryotic cells with the dyed T4 nanoparticles to obtain labeled eukaryotic cells.

In another embodiment, a method of labeling eukaryotic cells includes reacting T4 nanoparticles and dye molecules to produce dyed T4 nanoparticles, wherein reaction conditions favor the production of dyed T4 nanoparticles each comprising approximately n dye molecules, wherein n is a predetermined number between 100 and 20000; and contacting eukaryotic cells with the dyed T4 nanoparticles to obtain labeled eukaryotic cells.

A further embodiment includes an initial step of determining said quantity n by binding varying amounts of said dye to T4 nanoparticles to optimize n for maximum fluorescence.

In yet another embodiment, method of inducing protein expression in eukaryotic cells includes providing T4 nanoparticles comprising DNA encoding a protein, and contacting eukaryotic cells with the T4 nanoparticles thereby resulting in expression of the protein in the eukaryotic cells.

It is contemplated that the described T4 nanoparticles may also be used generally to deliver material to eukaryotic cells, by providing T4 nanoparticles comprising an exogenous material and contacting eukaryotic cells with the T4 nanoparticles to obtain eukaryotic cells comprising the exogenous material

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic of an exemplary approach for preparing a T4 nanoparticle covalently bound to dye molecules for probe synthesis. On the left is a representation of bacteriophage T4, on the right is a schematic representation of a tail-less T4 nanoparticle (“NP”) obtained by deletion of the tail using genetic engineering. As seen in the inset, in one embodiment free amine groups are used to incorporate fluorescent dyes using standard bioconjugation techniques.

FIG. 2 provides various UV-Vis spectra. FIG. 2A shows Cy3 NHS ester (black), T4 NP after conjugation with Cy3 (red), and T4 NP (blue). FIG. 2B shows Alexa 546 NHS ester (black), T4 NP after conjugation with Alexa 546 (red), and T4 NP (blue). Absorbance is normalized relative to dye concentration.

FIG. 3 shows fluorescence emission of dyed T4 NPs. Samples were excited at 550 nm and data were normalized relative to the number of unmodified T4 NPs. FIG. 3A is data from Cy3-T4 samples while FIG. 3B is from Alexa 546 samples.

FIG. 4 shows confocal microscopy images of A549 cells after uptake of dyed T4 NPs at different time points. Uptake of Alexa 546-dyed T4 NPs at 2282 dye mocules per viral capsid (“D/V”): (a) 4 h after incubation, (b) 8 h, (c) 24 h. Uptake of Cy3-T4 NPs (786 D/V) after incubation at (d) 4 h, (e) 8 h, and (f) 24 h. (g) Untreated cells, negative control. Imaging was performed under 60× magnification. The scale bar is 10 μm.

FIG. 5 shows flow cytometry histograms of A549 cells treated Cy3-T4 NP (786 D/V) on the left and Alexa 546-T4 NP (2282 D/V) on the right at different time points after incubation, 4 h (red), 8 h (blue), and 24 h (green); untreated cells after 24 h incubation are used as negative control (black line).

FIG. 6 shows flow cytometric detection of cell proliferation over time following Alexa 546-T4 NP treatment.

FIG. 7 shows delivery of fluorescent DNA into A549 cells through T4 NPs. A549 cells were treated with T4 NPs containing Alexa488 labeled DNA. FIG. 7A shows nuclei staining with 4′,6-diamidino-2-phenylindole (DAPI). FIG. 7B shows fluorescent T4 NPs and released fluorescent DNA (indicated by arrows). FIG. 7C is an overlay of FIGS. 7A and 7B. FIG. 7D is a brightfield image of the treated cells.

FIG. 8 shows an example of delivery of DNA for enhanced green fluorescent protein (eGFP) through T4 NPs and resulting protein expression. FIG. 8A shows eGFP expression after 48 hrs of the treatment with T4 NPs. FIG. 8B is a brightfield image of the same treated cells shown in FIG. 8A. FIG. 8C is a fluorescent image of the untreated cells, while FIG. 8D is a brightfield image of the same field as FIG. 8C. The images were obtained at 40× magnification. The scale bar is 10 μm.

FIG. 9 shows that intact T4 phage does not enter eukaryotic cells whereas T4 NPs enters eukaryotic cells. FIGS. 9A, 9B, and 9C are images of cells treated with the Alexa 488-phage. FIGS. 9D, E, F are the cell images treated with Alexa 488-T4 NPs. FIGS. 9A and 9D are brightfield images; FIGS. 9B and 9E are the fluorescent images; and FIGS. 9C and 9F are overlays of the respective brightfield and fluorescent images. The images were obtained at 40× magnification. The scale bar is 10 μm.

### DETAILED DESCRIPTION

Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used in this specification and the appended claims, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

As used herein, the term “T4 nanoparticle” refers to a non-infectious, tail-less variant of a T4 bacteriophage.

As used herein, the term “exogenous material” refers to material not naturally found in the T4 bacteriophage, including without limitation nucleic acids and/or protein.

When protein expression is referred to herein, it includes expression of small peptides as short as a single amino acid residue.

Description

T4 nanoparticles (“NPs”), that is non-infectious, tail-less variant of a T4 bacteriophage, can be produced in E. coli and manipulated genetically and/or chemically. For example, bacteriophage T4 DNA packaging is described in Lin et al., J. Biol. Chem. 272 (1997), 3495-3501.

T4 NPs have a relatively large capsid or “head” surface area which can accommodate many more functional groups than other icosahedral phages. The T4 capsid appears to be more flexible for surface functionalization than that of most other icosahedral viruses. Surface functionalization of T4 NPs for sensors, cellular probes, materials applications, and for delivery of material to eukaryotic cells is contemplated herein.

It is believed that the present invention includes the first demonstration that T4 nanoparticles can serve as agents to deliver exogenous material to eukaryotic cells.

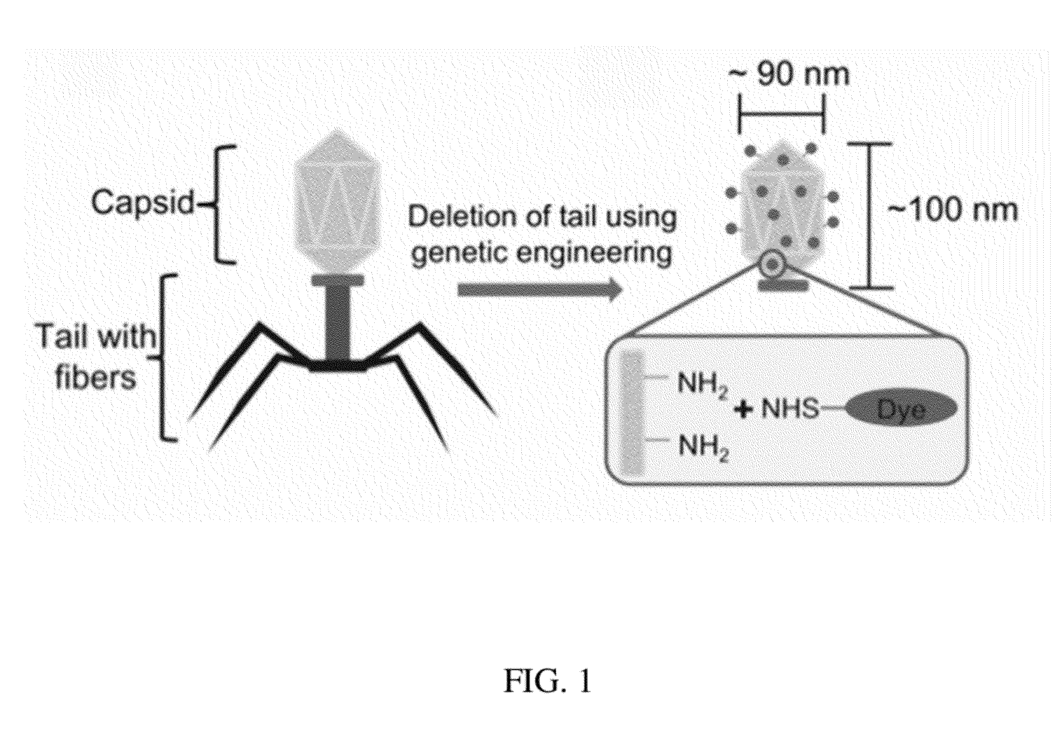

FIG. 1 is a schematic of an exemplary approach for preparing a T4 nanoparticle with dye molecules covalently bound thereto. On the left is a representation of bacteriophage T4, on the right is a schematic representation of a tail-less T4 nanoparticle (“NP”) obtained by deletion of the tail using genetic engineering. As seen in the inset, in one embodiment free amine groups are used to incorporate fluorescent dyes using standard bioconjugation techniques.

In a preferred embodiment, dye molecules are attached to T4 nanoparticles using N-hydroxysuccinimide esters (NHS ester) chemistry. Other suitable chemistries may be used for binding to amines found in T4 NPs, including imidoester, PFP ester, and hydroxymethyl phosphine.

A variety of dyes can be used. Preferably, fluorescent dye is used. In examples provided below, Cy3 and Alexa Fluor 546 (“Alexa 546”) serve as model dyes. These two fluorophores, while sharing similar excitation/emission spectra, have been reported to exhibit strikingly different performance characteristics, which are thought to be due, in part, to their molecular properties, but also to their neighboring interactions. Chemical bioconjugation of organic dyes has been utilized to prepare fluorescent probes using plant viruses such as cowpea mosaic virus (CPMV), turnip yellow mosaic virus (TYMV), tobacco mosaic virus (TMV), and potato virus X (PVX); however, there are fewer examples of the use of bacteriophages as scaffolds for this purpose. In plant viruses containing small capsids, such as CPMV and TMV (30 and 28 nm in diameter, respectively), the number of dyes that can be incorporated is limited by the surface area of the capsids and corresponding reactivities of solvent-accessible amino acids on the virus surface. Precise control of the spacing between the dyes becomes more critical within this limited surface area in order to avoid dimer formation and quenching of the dyes. While TMV with its “hollow-rod” shape has a larger surface area, the chemical conjugation is cumbersome, since commonly used residues, such as lysines, are not exposed on the surface. PVX on the other hand has an elongated capsid, and even with this significantly larger surface area, the maximum number of dyes incorporated is only 1600 per capsid through primary amine modification.

As described herein, more than 1.9×10⁴ dyes can be incorporated on a single T4 NP; this is believed to be the highest number of dyes incorporated onto a single viral nanoparticle. These results also show that the T4 probes are so efficient that even those with dye numbers as low as 350 dyes per particle give good fluorescence intensities for in vitro applications. In addition, the dyed T4 NPs can be used to visualize cells and remain inside cells for at least 72 hours.

These viral nanoparticles, using tail-less T4 heads, serves as scaffolds for dye attachment, and moreover, the resulting fluorescent Cy3-T4 and Alexa 546-T4 can serve as molecular probes for cellular imaging and flow cytometry. Large surface areas and protein contents of T4 NPs give the tail-less T4 NPs more functional groups and more flexibility for dye conjugation than other viruses. The structure and size of dyed T4 NPs are close to that of unlabeled T4 NPs, and the T4 NPs are intact and stable after conjugation and purification.

The described dyed T4 NPs conclusively demonstrate that T4 enters eukaryotic cells. Dye-T4 NPs can stay inside A549 cells at least 72 h and serve as ideal molecular probes for tracking cells, since cellular uptake of dyed T4 NPs can be clearly visualized by fluorescent microscopy, and cells positive for dyed T4 uptake can be distinguished and quantified by flow cytometry. The dyed T4 NPs can be used as a tool to understand in detail the molecular mechanisms of cell entry and the fate of the T4-NPs within cells.

Besides using dyed T4 nanoparticles for in vitro studies, it is also possible to use dyed T4 NPs as in vivo molecular probes, using chemical or genetic modification strategies for additional surface functionalization with target ligands and/or chemicals. The ability to load the substantial surface area of the T4 NPs with a large number of dyes per virus indicates that the surface can be loaded with a variety of functionalities by using the available reactive amines. This functionalization capability will be useful in vivo when adding labels such as poly(ethylene glycol) (PEG) to prevent blood clearance, target antigens to ensure delivery to desired targets (for example, to tumor tissues or infected cells such as those infected by a bioweapon), and/or dye labels.

As seen in the following examples, T4 nanoparticles have also been successfully used to deliver exogenous DNA for desired exogenous protein expression in eukaryotic cells by packaging exogenous DNA into T4 NPs in vitro.

It is further contemplated that T4 nanoparticles could be used for the delivery of other exogenous materials into eukaryotic cells. For example, one of ordinary skill in the art could insert DNA coding for one or more proteins of interest at the site of a T4 capsid protein such as Hoc and/or Soc to create a T4 nanoparticle carrying the protein(s) of interest on the surface. See Selick et al., J. Biol. Chem. 263(23) (1988) 11336-11347; Iwasaki et al., Virology 271:321-333 (2000); and US Patent Application Publication No. 2002/0025515. Alternately, or in addition, the T4 NP surface can be modified chemically, such as using chemistry described herein for dye attachment, to deliver protein, drugs, and/or other exogenous material to eukaryotic cells.

Preparing Purified Tail-Less T4 NPs

T4 K10 (38⁻51⁻denA⁻denB⁻), a kind gift from Dr. Lindsay Black at University of Maryland at Baltimore Medical school, was propagated in the suppressor E. coli host strain, CR63, to obtain infectious phage. The infectious phage was then used to produce non-infectious heads in the non-suppressor host E. coli strain Rosetta, essentially as described in Lin et al., J. Mol. Biol. 289(2) (1999), 249-60. In brief, Rosetta grown in M9S (OD₆₀₀=0.5) supplemented with 1/3 volume of Luria Broth were infected with K10 phage at a multiplicity of infection (MOI) of 3, followed by a repeated infection after 9 minutes and continued incubation for 2 hours at 37° C. Cells were spun down at 6,000×g for 10 minutes and resuspended in 10 mM potassium phosphate (pH 7.5) supplemented with 10 mM MgCl₂ and 2 mM CaCl₂, CHCl₃ (1/20 of total volume), DNaseI (40 μg/mL), RNase I (50 μg/mL), and one tablet of mini-EDTA-free complete protease inhibitor (Roche, Ind.) was then added to the cell suspension and the cells were gently shaken at 37° C. for 1-2 hours. The cell debris was removed after spinning for at least 15,000×g for 30 min. The cell lysate containing the head scaffolds was concentrated through Microcon YM-100 membrane according to the manufacturer's procedure (Millipore Corp, Mass.). The membrane was then washed 3-6 times with 10 mM potassium phosphate (pH 7.5) supplemented with 10 mM MgCl₂, followed by gel filtration with a column preloaded with Superose 6 (GE Healthcare Biosciences, N.J.). The flow-through and eluted fractions were then assessed using 0.8% Tris-Acetate agarose gel stained with either ethidium bromide or Coomassie blue stain. Fractions were obtained having no detectable DNA contamination and no detectable protein contamination.

This preparatory method using gel filtration can easily scale up to larger production volumes at greatly reduced cost compared to the traditional method using ultracentrifugation.

Preparation of T4 NPs Bound to Dye

Tail-less T4 NPs (here we use K10 full head containing DNA and referred as K10 head) were suspended in 50 mM potassium phosphate buffer (KP), pH 7.5 supplemented with 10 mM MgCl₂. Dye conjugation reactions were done in the same buffer condition in the presence of 10% dimethylsulfoxide (DMSO) (Sigma-Aldrich, St. Louis, Mo.) at 22° C. for 16 hrs. Virus concentration was determined on the basis that 1 mg corresponds to 9.7×10¹² heads (based on the copy numbers of proteins assembled on the T4 heads). In a typical reaction, 4.5×10¹¹ particles (approximately 46 μg) of T4 NPs were used. Varying molar ratios of reactive dye to T4 nanoparticle were employed in various reactions to obtain dyed T4 NPs having different ratios of dye to virus (the ratio termed “D/V”).

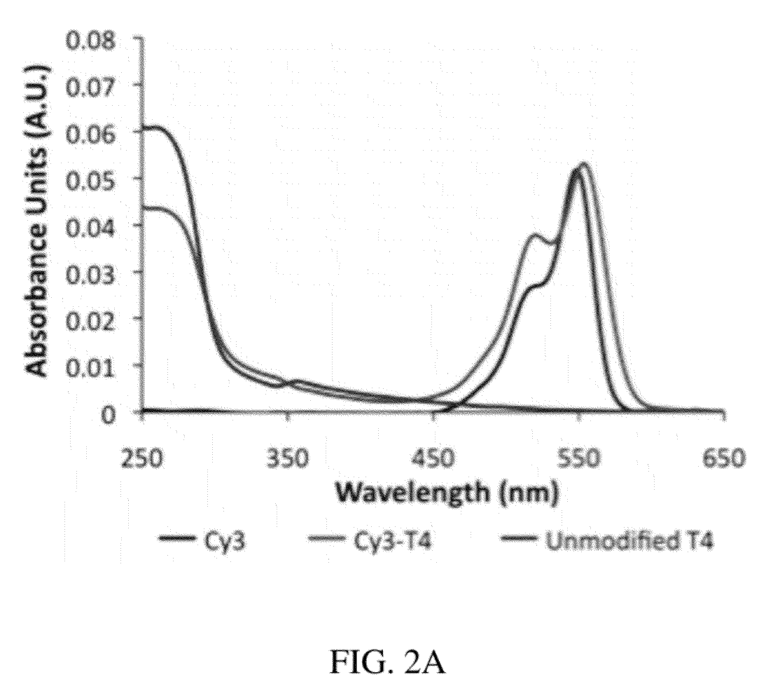

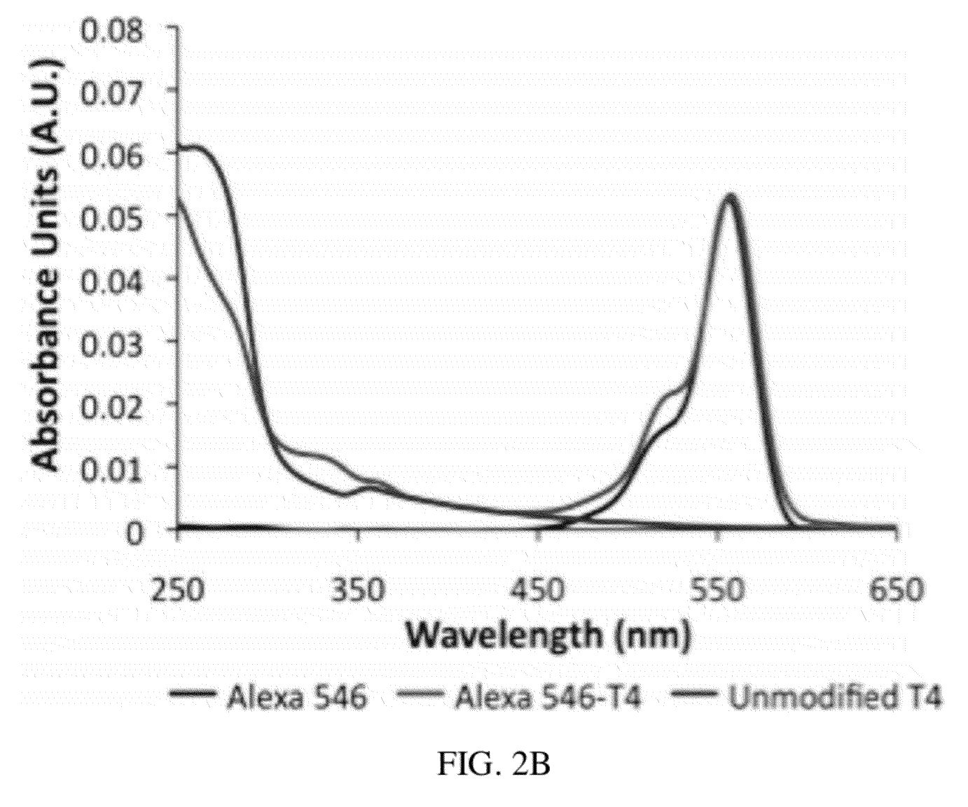

Dye-T4 NPs mixtures were loaded separately in a Superose™ 6 prep grade (GE Healthcare Biosciences) column pre-equilibrated in 50 mM KP/10 mM MgCl₂ with the addition of 0.05% sodium azide). Individual 1 ml elutions were collected and analyzed by UV-Vis spectroscopy (FIG. 2). Elutions were analyzed by UV-Vis Spectroscopy (Cary 5000, Varian). Cy™ 3 Mono NHS ester (NHS-Cy3; GE Healthcare Biosciences) was used in the reaction. Dye per virus (D/V) values were calculated by using the extinction coefficient provided by the manufacturer, i.e.,150000 M⁻¹ cm⁻¹ for Cy3, to determine dye concentration and virus concentration was calculated from virus absorbance at 260 nm. Cy3-T4 NPs containing fractions were analyzed by fluorescence spectroscopy and agarose electrophoresis.

Conjugation reactions carried out in buffer alone resulted in an average of 10× lower reactivity in comparison to reactions carried out in 90:10 buffer to DMSO, due to the enhanced solubility of the dye in DMSO. In addition, including MgCl₂ (1, 2, or 10 mM) in the reaction buffer enhanced the stability of T4 and did not affect the reactivity of the dyes. In early optimization reactions, fluorescein-5-isothiocyanate (FITC) and Alexa Fluor 488 sulfodichlorophenol ester (Alexa488-SDP ester) were compared to determine the level of reactivity of the T4 NP amines in different buffer conditions. Both dyes have similar spectral characteristics, which facilitates the comparison of UV-visible data when determining number of dyes per T4 NP. FITC is known to provide reasonable specificity toward the ε-amine of lysine, while Alexa 488-SDP ester reacts with all primary amines in a similar manner as common NHS-ester dyes. By comparing FITC with Alexa 488-SDP, one can derive conclusions about the availability of amines on the T4 surface.

Characterization of Dyed T4 NPs

In addition to the gel analysis described above, atomic force microscopy (AFM) topographical images were used to examine the structural features of the unlabeled and dye-labeled T4 NPs. The T4-NPs were found to have defined shapes and borders and be well dispersed. Cross-sectional analysis found that the average length of the unlabeled nanoparticle is 169±14 nm, which is larger than the predicted length of 119.5 nm from a cryo-EM model. Its average height is 39±3 nm, half of what was predicted from cryo-EM. The difference in length is mainly due to both virus collapsing and AFM tip effect; however, the differences in height of the T4 NPs prepared here can be explained by the loss of DNA from the NP during preparation. According to solution spectroscopic analysis of the T4 NPs, 10% of the capsids still contain DNA. The empty NPs are collapsed to half of the original height during drying for AFM measurements.

Overall, the height and length measurements of the unlabeled and dyed T4 NPs are comparable. The observed shape and close values of the average length and height between the unmodified and the modified T4 NPs indicate that the capsid withstands the conditions used for the chemical conjugation and purification. This is relevant to the possible effects of dimethylsulfoxide (DMSO), which can denature proteins. It was found that increasing the amount of DMSO beyond about 10% resulted in debris observed under AFM.

Spectroscopic Properties of Dye-T4 NPs

UV-Vis measurements were taken of the Cy3-T4 and Alexa-T4 NPs and compared to spectra from the free dyes and unlabeled T4 NPs, as seen in FIG. 2. The 260-280 nm peaks in the T4 spectra represent the absorbance of the viral genomic DNA inside the T4 NP and the capsid proteins, respectively, while the peaks in the visible region represent the dye absorbance. There is a small red shift (5 nm) in absorption maxima of the Cy3 upon conjugation to the T4 NP, which is known to indicate cyanine dye-protein coupling. Alexa 546-T4 NPs also show a slight shift in the dye absorbance compared to free Alexa 546, which is typical of other Alexa fluorophores.

Dye per Virus (D/V) Measurements

Absorbance measurements taken at the dye wavelengths for each dyed T4 NP sample along with the number of NPs present in each sample were used to calculate the D/V. As the molar excess of dyes to lysines was increased, the D/V increased up to 1.9 ×10⁴ Alexa 546 dyes obtained under reaction conditions of 20 dyes per T4 NP lysine. See Robertson et al., “Engineered T4 viral nanoparticles for cellular imaging and flow cytometry” Bioconjugate Chem. 2011, 22, 595-604, and accompanying Supporting Information, both incorporated by reference herein. This D/V ratio is two orders of magnitude higher than other icosahedral viruses, such as CPMV and one order of magnitude higher than the maximum D/V on the rod-shaped potato virus X and tobacco mosaic virus. This high degree of labeling is possible due to the large number of amine groups present on the T4 NP.

Fluorescence Properties of Dye-T4 NPs as a Function of D/V

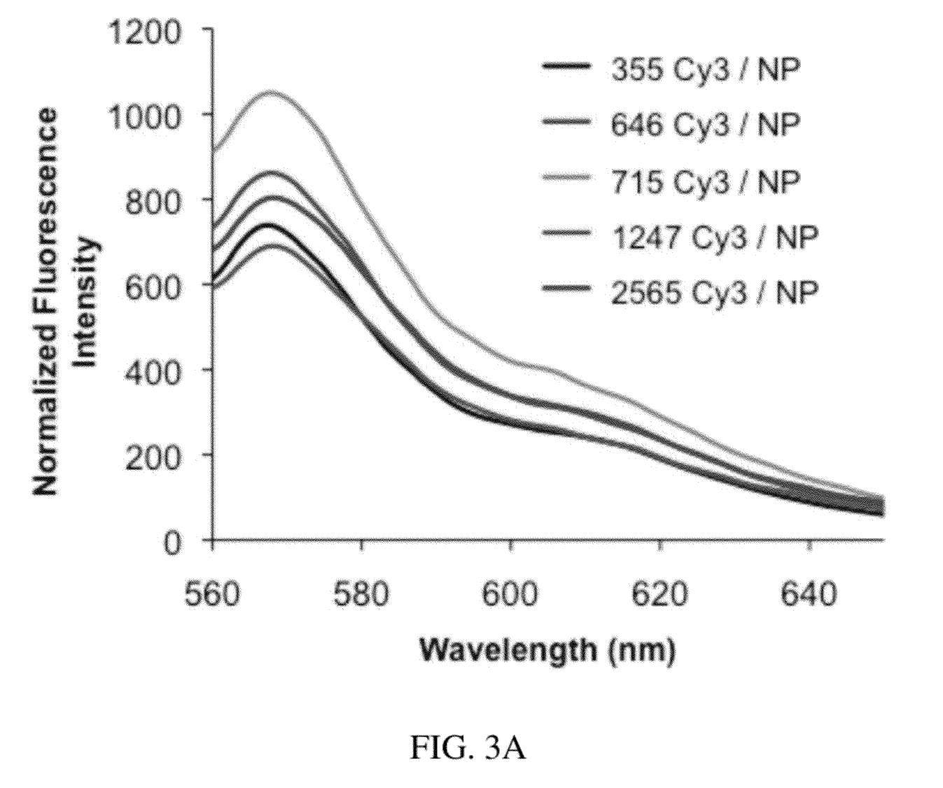

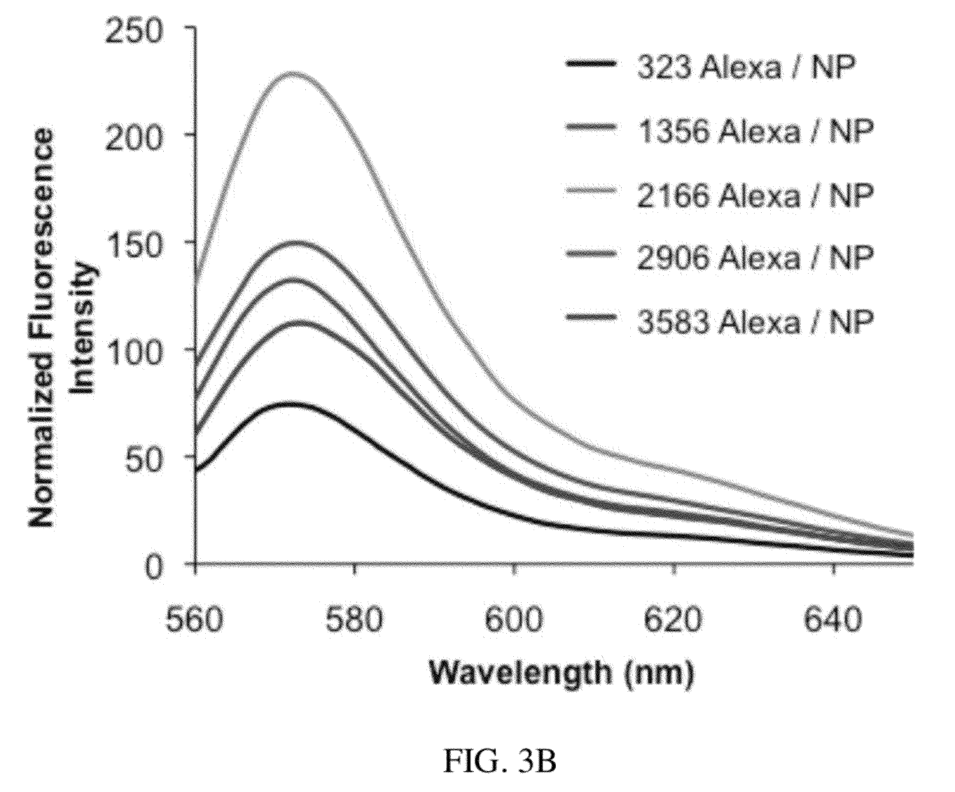

Fluorescence emission of the dyed T4 complexes was studied as a function of D/V. As shown in FIG. 3A, fluorescence emission increases as the number of dyes in the Cy3-T4 is increased up to 715 D/V (the orange line in FIG. 3A); beyond that point, fluorescence decreases as the number of dyes increases presumably due to quenching. In contrast, for the Alexa 546-T4 series depicted in FIG. 3B, the best fluorescence output is obtained at 2166 D/V (also the orange line).

Dye-T4 NPs as Molecular Probes

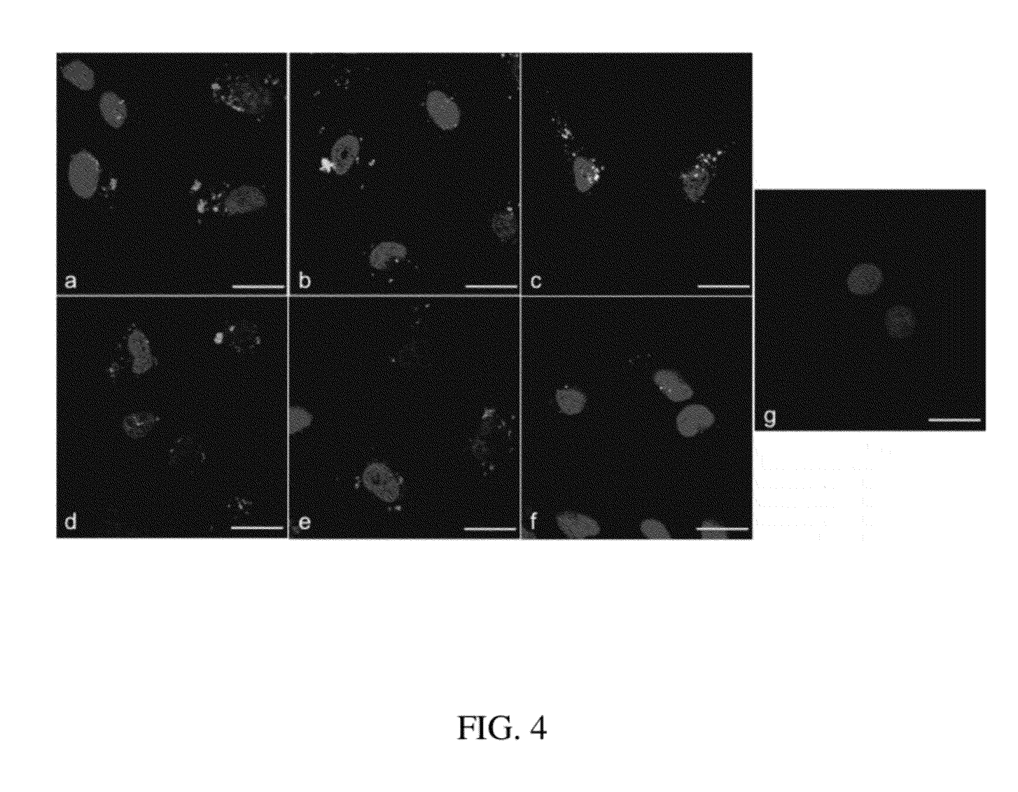

The high fluorescence output and biocompatibility of dyed T4 NPs make them suitable as molecular probes for cellular imaging and flow cytometry. To explore their application as molecular probes, cellular uptake was explored using the lung cancer cell line, A549. Confocal microscopy was used to qualitatively investigate the interaction between the dyed T4 NPs and A549 cells at different incubation times, seen in FIG. 4. A549 cells were treated with Cy3-T4 (786 D/V) or Alexa 546-T4 NPs (2282 D/V) at a ratio of 1 cell to 105 T4 NPs. The nuclei were visualized by DAPI staining (blue), and the dyed T4 NPs are shown as small yellow spots distributed though the cell interior. Both types of dyed T4 NPs, Cy3-T4 and Alexa 546-T4 NPs, were clearly visible inside cells after 4, 8, and 24 h (FIGS. 4A-F). Untreated cells are in FIG. 4G.

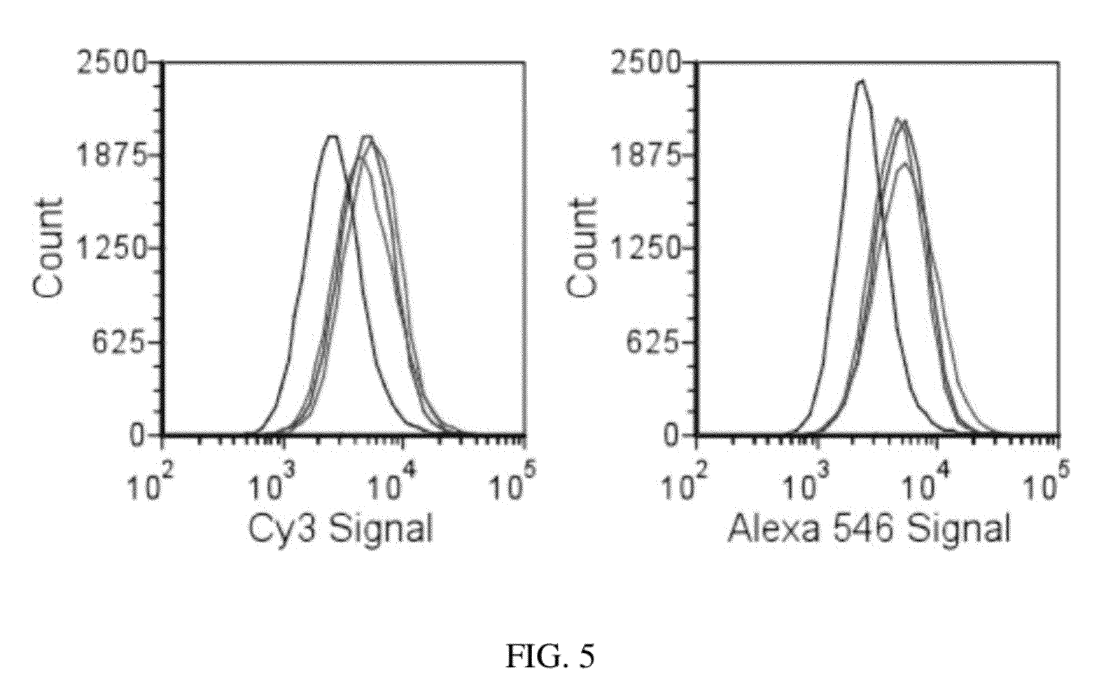

Cellular uptake was also explored by flow cytometry, which can be used to separate and quantitatively measure fluorescently labeled cell populations using different molecular probes. The change in fluorescence between the dyed T4 NP treated cells and the untreated cells was used to measure uptake. FIG. 5 shows that a clear shift is present when cells are treated with both dyed T4 NPs in comparison to untreated cells. Furthermore, the results from the flow cytometry measurements were used to quantitatively measure the uptake of the dyed T4-NPs into cells at different time points. The percentage of cells positive for dyed T4 NPs was determined by subtracting the treated histograms from the untreated. These results indicate that the percentage of cells that are positive for dyed T4 NPs is greater than 50% at all time points, suggesting that the uptake of T4 NP is quite efficient. Flow cytometry was further used to determine the cell viability after cellular uptake of dyed T4 NPs by staining both treated and untreated cells with a live/dead stain. More than 92% of the cells containing the dyed T4 NPs were alive compared with >95% of untreated cells. This suggests that dyed T4 NPs have low cytotoxicity, even after 24 h. The ability to use dyed T4 NPs for flow cytometry opens the possibility of a number of applications including cell tracking, mechanistic studies, and immunoassays.

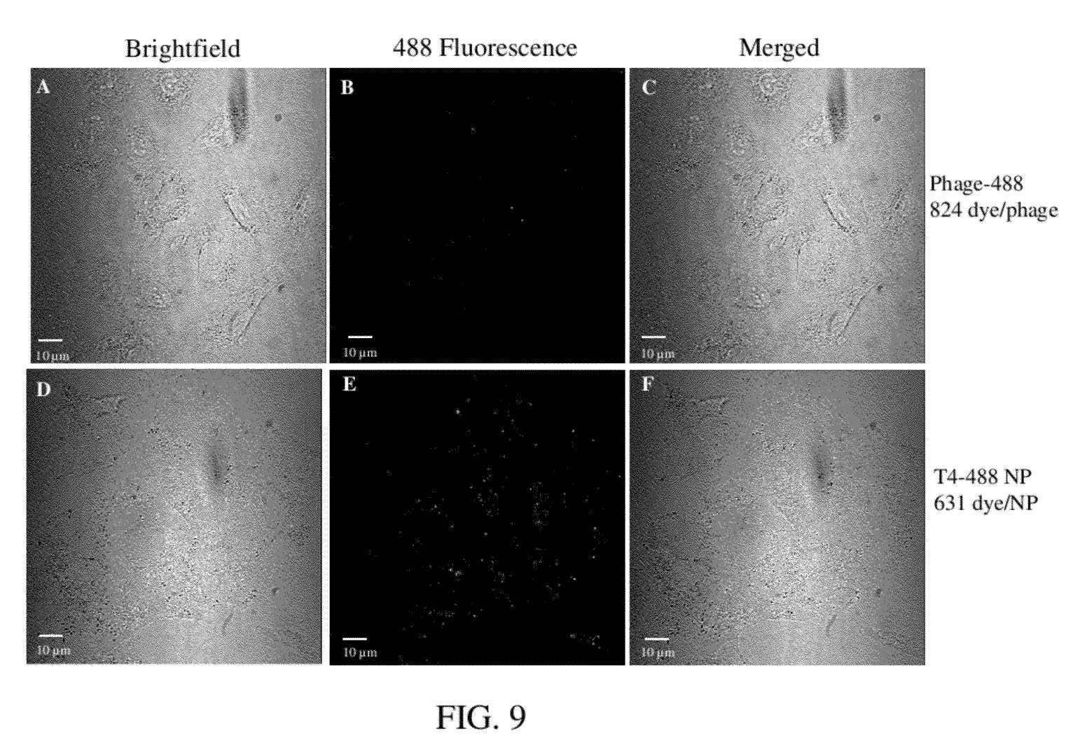

In addition to the above-described examples using A549 lung cancer cells, T4 NPs were also found to enter the interior of noncancerous primary cultured cells in vitro, including human endothelial cells, liver cells, and murine astrocytes. Thus, it is expected that T4 NPs should be internalized by eukaryotic cells. However, as described below and in FIG. 9, it was surprisingly found that dyed T4 phage (including the bacteriophage tail) was not internalized by eukaryotic cells.

Cell Tracking

The ability to label cells and/or track cell proliferation can be useful in a variety of in vitro and in vivo studies. To explore this application for T4 NPs, A549 cells were treated with Alexa 546-T4 (2962 D/V) for 6 h and allowed the cells to grow for up to 96 h following treatment. A cell counter equipped with a fluorescence module was used to measure the number of cells and percentage of cells positive for T4 NPs. It was found that as the cells divided and the number of total cells increased, the percentage of cells positive for Alexa 546-T4 NP decreased. After 96 hours, the percentage of cells positive for Alexa 546-T4 NPs decreased to 1.4%, a level equal to the background from untreated cells. When comparing the treated cells to untreated cells, there is no significant decrease in proliferation in dyed T4 NP treated cells, indicating a lack of toxicity.

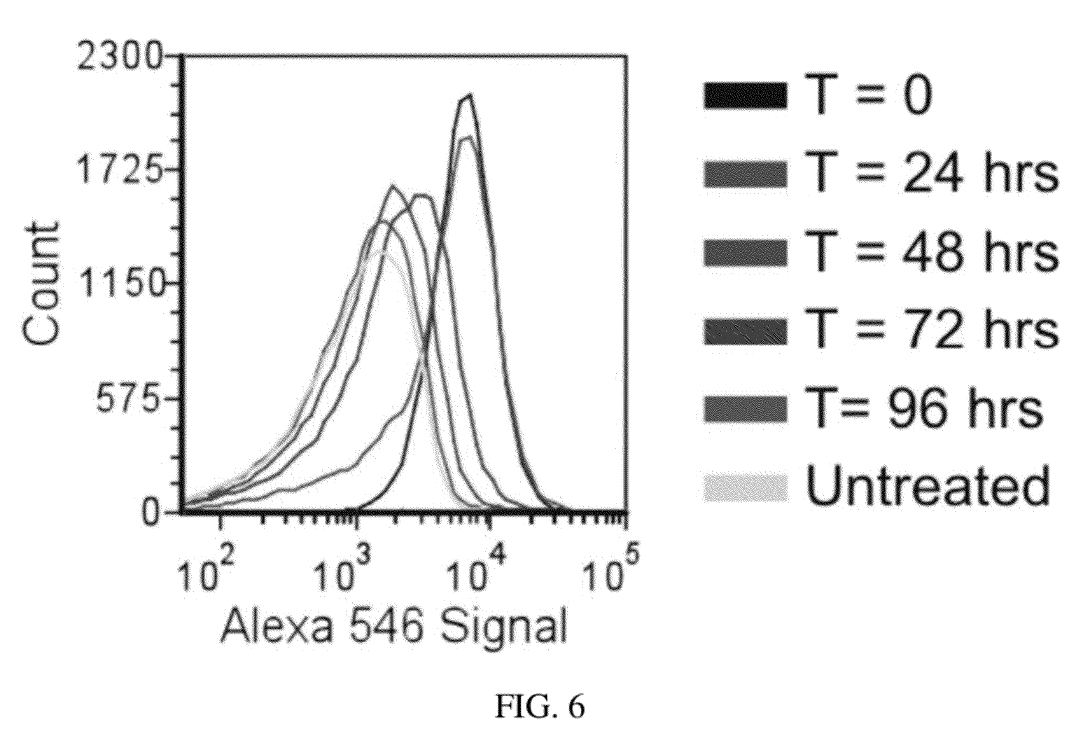

The fluorescence of the cells was also measured by flow cytometry. The data obtained and seen FIG. 6 shows that, over time, as the cells proliferate, the fluorescence of the cell population decreases and the population becomes more heterogeneous. This supports the hypothesis that, over time, the fluorescence signals will decrease due to the dilution of Alexa 546-T4 NPs to newly divided cells. These results demonstrate that the internal dyed T4 can remain inside A549 cells for at least 72 hours and that dyed T4 NPs can be used to quantitatively track cellular proliferation.

DNA Delivery for Protein Expression

In view of the finding that T4 nanoparticles enter eukaryotic cells, it was contemplated that T4 nanoparticles could be used to deliver exogenous double-stranded (“ds”) DNA. Such delivery could be useful for expression of desired proteins in cell culture (transfection) and possibly in living organisms for gene therapy. Other uses for this technique include the delivery of pharmaceutical compounds. Delivery may be targeted by including appropriate surface modification of the T4 NPs.

To first determine whether a T4 NP could deliver DNA into a eukaryotic cell, double stranded DNA (dsDNA) was first prepared and labeled with fluorescent dye. One of ordinary skill in the art can conduct reverse transcription on suitable mRNA or dsRNA using conventional techniques, to produce labeled or non-labeled DNA. To prepare labeled DNA in this example, a reverse transcription reaction was conducted using 0.5 mM d-ATP, 0.5 mM d-GTP, 0.5 mM d-TTP and a mixture of 0.1 mM d-CTP and 0.2 mM 5-aminohexylacrylamido-dCTP (aha-dCTP) (a 1:2 ratio) to produce amine-modified DNA, which can then be labeled with an amine-reactive dye or hapten.

Three Pseudomonas phage phi-6 dsRNA genome fragments, L, M, and S (NC003714, NC003715, NC003716) were isolated and used for reverse transcription. Three primer pairs, RT-S F1(GTGTTGTTCCCACTAATAAT), SEQ ID No: 1; RT-S R1 (ATGCATGAAGGGGCTGGAC), SEQ ID No: 2; RT-M F1 (TCAACCCATAATAAGAGATC), SEQ ID No: 3, RT-M R1(CATCCTATTGGATGCTCGC), SEQ ID No: 4; RT-L F1 (ATCCGACTTTTATAAGGACG), SEQ ID No: 5; and RT-L R1 (TGATGTTACCAACGAAGATG), SEQ ID No: 6 were used to reverse transcribe two complementary phi-6 S, M, L cDNAs at 47° C. for one hour, followed by denaturing at 85° C. and gradually cooling down to 4° C. to make three dsDNA, 1520 bp, 3320 bp, 2030 bp, in length, respectively. The resulting dsDNAs (˜2 μg) were then precipitated by ethanol at −80° C. and were dissolved in 5 μL H₂O, followed by the addition of 2 μL of the reactive dye NHS-Alexa 488 at the concentration of 30 μg/μL and 3 μL of the sodium bicarbonate (80 mg/mL). The reaction was then incubated at 22° C. for 1 hr using MJ research Tetrad Thermo Cycler (MJ research Inc, Waltham, Mass.). The fluorescent DNA was then characterized using a mass spectrometer and examined using gel electrophoresis. The fluorescent DNA was packaged into T4 NPs in vitro using T4 phage components as described in Black and Peng, J. Biol. Chem., (2006) Vol. 281, No. 35, pp. 25635-25643. As noted below, the T4 NPs were effective in delivering the fluorescent DNA into eukaryotic cells.

To test whether application of the T4 nanoparticles loaded with DNA would result in protein expression, T4 NPs were prepared containing eGFP (enhanced green fluorescent protein). Here, the eGFP expression plasmid, peGFP-N1, was obtained from Clontech Laboratories (Mountain view, Calif.). The plasmid was linearized using ApaL1 without interrupting the CMV promoter for RNA transcription. Other DNA containing a eukaryotic gene expression promoter, such as CMV and encoding a desired protein can be used.

The linearized DNA was separated on agarose gel and purified using Qiagel purification kit, followed by Qiaquick purification (Qiagen Inc, Valencia, Calif.). Purified DNA was packaged according to Black and Peng (2006). See also Zhang Z., et al. (2011) “A Promiscuous DNA Packaging Machine from Bacteriophage T4.” PLoS Biol 9(2): e1000592.

T4 NPs packaged with either dye-labeled DNA or linearized peGFP-N1 DNA were added to A549 cells at an estimated ratio of 50,000 particles per cell, for 15-18 hrs. Cells treated with fluorescent T4 NPs were then fixed with 4% formaldehyde for 15 min, followed by staining with 5 μg/mL of 4′,6-Diamidino-2-phenylindole dihydrochloride (DAPI) (Sigma-Aldrich, St. Louis, Mo.) at RT for 15 min. The cells were washed with PBS at least three times after each staining. Cells treated with T4 NPs containing peGFP DNA were further incubated with the growth medium for 48 hrs after washing off the T4 NPs. Cells were fixed with DAPI staining as previously described. The images were obtained using epifluorescent microscope (Nikon TE2000) under the magnification of 60× and analyzed using the Nikon Imaging software, NIS-Elements AR 3.10.

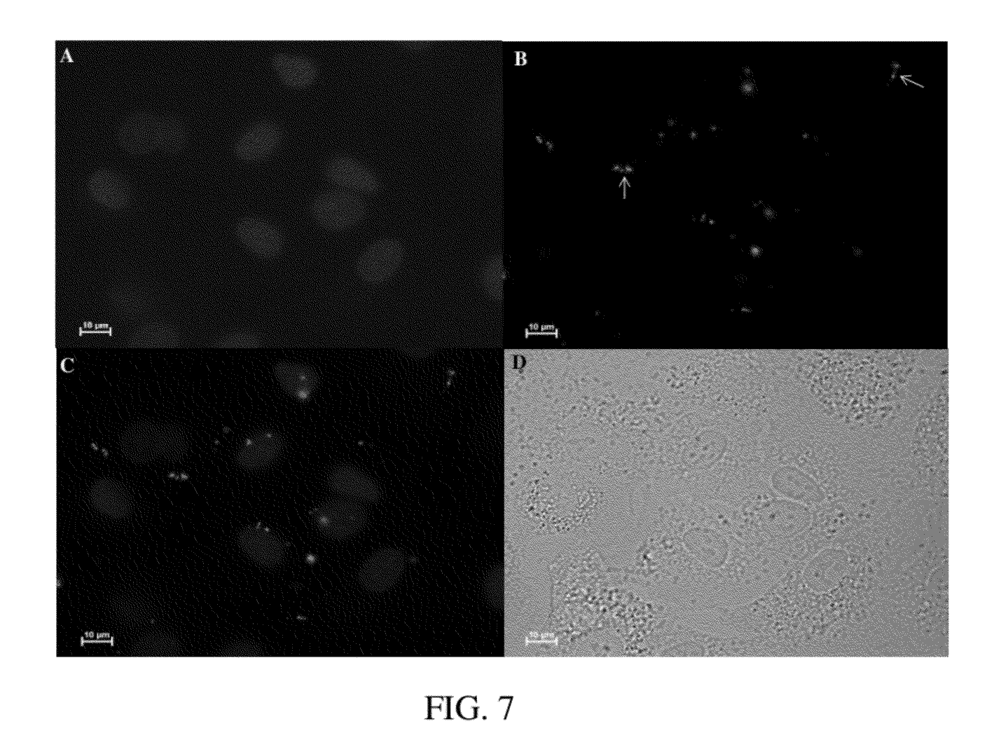

FIG. 7 shows delivery of fluorescent DNA into A549 cells through T4 NPs. A549 cells were treated with T4 NPs containing Alexa488 labeled DNA prepared as described above. FIG. 7A shows nuclei staining with DAPI. FIG. 7B shows fluorescent T4 NPs and released fluorescent DNA (indicated by arrows). FIG. 7C is an overlay of FIGS. 7A and 7B. FIG. 7D is a brightfield image of the treated cells.

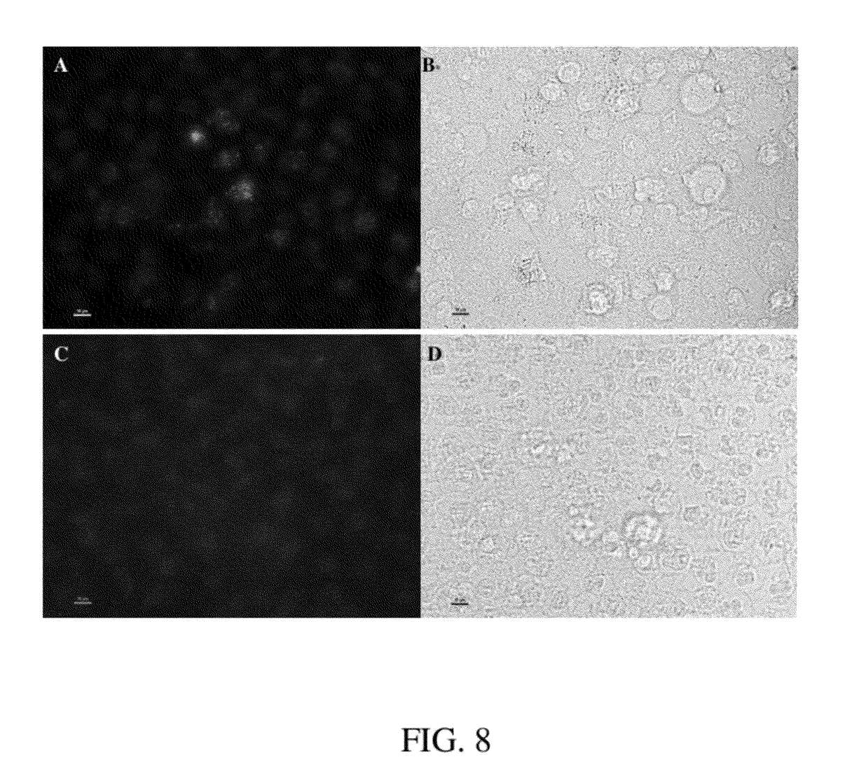

FIG. 8 shows an example of delivery of enhanced GFP protein through T4 NPs and resulting protein expression. A549 cells were treated with T4 NPs containing linearized peGFP DNA. FIG. 8A shows eGFP expression after 48 hrs of the treatment with T4 NPs. FIG. 8B is a brightfield image of the same treated cells in the field shown in FIG. 8A. FIG. 8C is a fluorescent image of the untreated cells, while FIG. 8D is a brightfield image of the same field as FIG. 8C. The images were obtained at 40× magnification. The scale bar is 10 μm.

Lack of Cellular Delivery of Intact T4 Phage

Although T4 NPs (which lack the bacteriophage tail) were found to be internalized by eukaryotic cells, it was surprisingly found that T4 phage (which includes the bacteriophage tail) was not internalized by eukaryotic cells, as seen in FIG. 9.

T4 phage was purified using the procedure described above for the preparation of T4 NPs with the exception that the E. coli strain CR63 was used. Alexa488 was used for labeling T4 phage and NPs as described above. There were 824 dyes/phage and 631 dyes/NP. A549 cells were treated with either of the labeled materials using a ratio of 100,000 dyed particles per cell for 4 hours. FIGS. 9A, 9B, and 9C are images of cells treated with the Alexa488-phage. FIGS. 9D, E, F are the cell images treated with Alexa 488-T4NPs. FIGS. 9A and 9D are brightfield images; FIGS. 9B and 9E are the fluorescent images; and FIGS. 9C and 9F are overlays of the respective brightfield and fluorescent images. The images were obtained at 40× magnification.

Concluding Remarks

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

All documents mentioned herein are hereby incorporated by reference in their entireties.

What is claimed is:

1. A method of labeling eukaryotic cells, the method comprising:
providing dyed T4 nanoparticles, wherein each dyed T4 nanoparticle comprises at least 350 dye molecules covalently bound thereto, and
contacting eukaryotic cells with the dyed T4 nanoparticles to obtain labeled eukaryotic cells.

2. The method of claim 1, wherein said dye is a fluorescent dye.

3. The method of claim 1, wherein each T4 nanoparticle is bound to at least 1000 dye molecules.

4. The method of claim 3, wherein said T4 nanoparticle comprises at least 5000 dye molecules.

5. The method of claim 1, further comprising an initial step of purifying T4 nanoparticles via gel filtration.

6. The method of claim 1, wherein T4 nanoparticles are obtained without the use of ultracentrifugation.

7. The method of claim 1, further comprising:
synthesizing said dyed T4 nanoparticles by reacting T4 nanoparticles and dye molecules, wherein reaction conditions favor the production of dyed T4 nanoparticles each comprising approximately n dye molecules, wherein n is a predetermined number between 100 and 20000.

8. The method of claim 7, wherein said reacting comprises reacting in the presence of DMSO and water.

9. The method of claim 7, further comprising an initial step of determining said quantity n by binding varying amounts of said dye to T4 nanoparticles to optimize n for maximum fluorescence of said dyed T4 nanoparticles.

10. A method of inducing protein expression in eukaryotic cells, the method comprising:
providing T4 nanoparticles comprising exogenous DNA encoding a protein, and
contacting eukaryotic cells with the T4 nanoparticles thereby resulting in expression of the protein in the eukaryotic cells.

11. The method of claim 10, wherein said eukaryotic cells are in vitro.

12. The method of claim 10, wherein said eukaryotic cells are in vivo.

13. A method of delivering material to eukaryotic cells, the method comprising:
providing T4 nanoparticles comprising an exogenous material and
contacting eukaryotic cells with the T4 nanoparticles to obtain eukaryotic cells comprising the exogenous material.

14. The method of claim 13, wherein said eukaryotic material comprises exogenous DNA.

15. The method of claim 13, wherein said eukaryotic material comprises a protein modification of a T4 capsid protein.
